(19) United States
(12) Patent Application Publication
(10) Pub. No.: US 2025/0302634 A1
(43) Pub. Date: Oct. 2, 2025

(54) Interbody Graft Containment Feature for Spinal Implants

(71) Applicant: K2M Inc., Leesburg, VA (US)

(72) Inventor: Jordan Holder, Chester, NY (US)

(21) Appl. No.: 19/057,309

(22) Filed: Feb. 19, 2025

Related U.S. Application Data
(60) Provisional application No. 63/571,540, filed on Mar. 29, 2024.

(51) Int. Cl. A61F 2/44; A61F 2/28; A61F 2/30

(52) U.S. Cl. CPC A61F 2/4455; A61F 2002/2835; A61F 2002/30136; A61F 2002/30593; A61F 2002/30784

(57) ABSTRACT

The present disclosure is directed to medical implants for better retention of biological bone graft material. The implants according to the present disclosure include an undulating crossbeam within a graft chamber to increase surface area and friction for retention of biological graft material above and below the crossbeam. In certain embodiments, implants can have smooth and curved sinusoidal peaks or sharp and planar sinusoidal peaks within the graft chamber.

### CROSS-REFERENCE TO RELATED APPLICATION

This application claims priority to the filing date of U.S. Provisional Patent Application No. 63/571,540, filed Mar. 19, 2024, the disclosure of which is hereby incorporated herein by reference.

### BACKGROUND

The present disclosure relates to medical implants for use in surgical procedures. While largely discussed in connection with use in spinal implants, the present disclosure is also applicable to other types of orthopedic and medical implants.

Common maladies of the spine result from a degeneration of the disc space.

This typically results from the degradation of the spinal disc, which in turn impacts the spacing between vertebral bodies. Spine fusion procedures have been developed to address these issues. Whether involving the use of pedicle screws and/or an interbody device (“IBD”), the goal of such a procedure is the fusion of adjacent vertebral bodies. This often results in long-term pain relief for a patient. Moreover, if done properly, a spinal fusion procedure is a long-term solution to pain resulting from improper vertebral body spacing.

To ensure a solid fusion between vertebral bodies, IBDs are conventionally packed with biological graft material before inserting the IBD into a patient. This material facilitates bone growth through the IBD to provide a secure connection between the implant and bone, and in essence, the adjacent vertebral bodies. Existing IBD designs often suffer from the inadvertent and unwanted displacement of biological graft materials during the transition from a sterile field into a patient. This displacement can be caused by low biological graft material viscosity, force of gravity, impaction forces, and/or unintended movement, such as bumping. Conventional methods of retaining biological graft material require using additional instrumentation to retain the biological material or require re-packing the graft chamber with more biological graft material. All of this adds to the complexity of spinal fusion surgeries and extends the time taken to conduct same.

Accordingly, there is a need for spinal implants with improved biological graft material retention.

### BRIEF SUMMARY

The present disclosure relates to different spinal interbody implants (IBDs) with structure that facilitates better retention of biological graft material. For instance, the IBDs disclosed herein include undulating crossbeams to increase surface area and friction. As a result, these IBDs will have improved case of use and should result in a strong connection to vertebral bodies above and below the IBD.

In one embodiment, an IBD has a graft chamber extending from a superior side of the IBD to an inferior side of the IBD. The IBD also has a crossbeam located within the graft chamber. The crossbeam has an undulated surface with a plurality of peaks facing at least one of the superior or inferior sides of the IBD.

In some examples, the plurality of peaks are configured to increase surface area and friction between the IBD and a biological graft material.

In some examples, the plurality of peaks are smooth and curved sinusoidal peaks. In other examples, the plurality of peaks are sharp and planar sinusoidal peaks. In other examples, the plurality of peaks are smooth and curved sinusoidal peaks and sharp and planar sinusoidal peaks.

In some examples, the IBD has a series of undercuts on a top and a bottom surface of the crossbeam. The series of undercuts substantially face a posterior side of the IBD.

In some examples, the plurality of peaks are of differing heights. In some examples, the plurality of peaks extend along a direction substantially parallel to an anterior side and a posterior side of the IBD. In some examples, the plurality of peaks extend along a direction substantially parallel to a first and second lateral side of the IBD.

In some examples, the crossbeam has a plurality of channels extending from the undulated surface to a second surface of the crossbeam. In some examples, the plurality of channels are of differing diameters.

In another embodiment, an IBD has a graft chamber and a crossbeam having two undulating surfaces facing opposing directions. The crossbeam extends from an anterior side of the graft chamber to a posterior side of the graft chamber.

In some examples, the opposing directions are towards a superior side of the IBD and an inferior side of the IBD.

In some examples, the crossbeam has a plurality of channels extending between the two undulated surfaces.

In yet another embodiment, an IBD has a graft chamber and a crossbeam having a first undulated surface facing a superior side of the IBD and a second undulated surface facing an inferior side of the IBD.

In some examples, the crossbeam has a plurality of channels extending through the first and second undulated surfaces.

In some examples, the IBD has biologic graft material in the graft chamber. In some examples, the biologic graft material is between the first undulated surface and the superior side of the IBD. In some examples, the biologic graft material is between the second undulated surface and the inferior side of the IBD.

In some examples, the IBD has an outer wall defining at least a portion of a perimeter of the IBD.

### BRIEF DESCRIPTION OF THE DRAWINGS

FIG. 1 is a top perspective view of a spinal implant in accordance with an embodiment of the present invention.

FIG. 2 is a front perspective view of the implant of FIG. 1.

FIG. 3 is a top plan view of the implant of FIG. 1, with the bottom plan view being a mirror image thereof.

FIG. 4 is a rear perspective view of the implant of FIG. 1.

FIG. 5 is a right-side plan view of the implant of FIG. 1, with the left-side perspective view being a mirror image thereof.

FIG. 6 is a cross-sectional view of the implant of FIG. 1 taken along line A-A of FIG. 3.

FIG. 7 is a top perspective view of a crossbeam of the implant of FIG. 1, with the bottom perspective view being a mirror image thereof.

FIG. 8 is a top perspective view of another spinal implant in accordance with an embodiment of the present invention, with the bottom perspective view being a mirror image thereof.

FIG. 9 is a top plan view of the implant of FIG. 8, with the bottom plan view being a mirror image thereof.

FIG. 10 is a rear perspective view of the implant of FIG. 8.

FIG. 11 is a front perspective view of the implant of FIG. 8.

FIG. 12 is a cross-sectional view of the implant of FIG. 8 taken along line A-A of FIG. 9.

FIG. 13 is a top perspective view of a crossbeam of the implant of FIG. 8, with the bottom perspective view being a mirror image thereof.

FIG. 14 is a top perspective view of another spinal implant in accordance with an embodiment of the present invention.

FIG. 15 is a front perspective view of the implant of FIG. 14.

FIG. 16 is a top plan view of the implant of FIG. 14, with the bottom plan view being a mirror image thereof.

FIG. 17 is a rear perspective view of the implant of FIG. 14.

FIG. 18 is a right-side plan view of the implant of FIG. 14, with the left-side perspective view being a mirror image thereof.

FIG. 19 is a cross-sectional view of the implant of FIG. 14 taken along line A-A of FIG. 16.

FIG. 20 is a top perspective view of a crossbeam of the implant of FIG. 14, with the bottom perspective view being a mirror image thereof.

### DETAILED DESCRIPTION

Particular embodiments of the present disclosure will be described herein with reference to the accompanying drawings. As shown in the drawings and as described throughout the following description, and as is traditional when referring to relative positioning on an object, the term “proximal” should be understood as referring to the portion of a structure that is closer to a clinician during proper use and the term “distal” should be understood as referring to the portion of a structure that is farther from the clinician during proper use. Also, as used herein, the terms “substantially,” “generally,” and “about” are intended to mean that slight deviations from absolute are included within the scope of the term so modified. Wherever possible, and for the sake of brevity, the same or like reference numbers will be used throughout the drawings to refer to the same or like features within a different series of numbers (e.g., 100-series, 200-series, etc.).

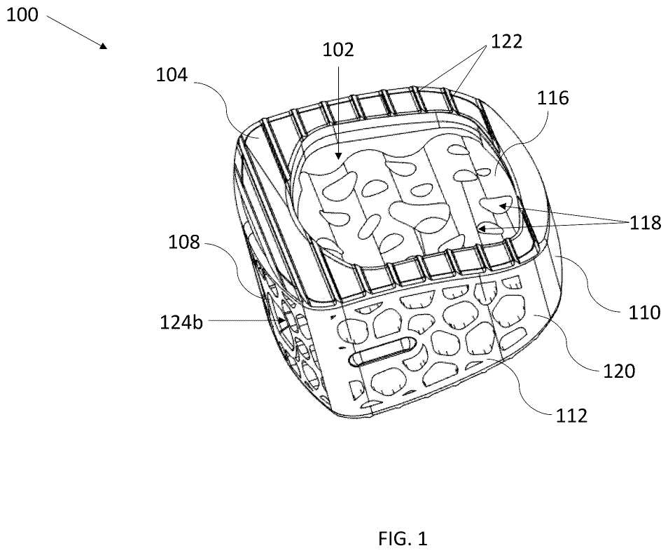

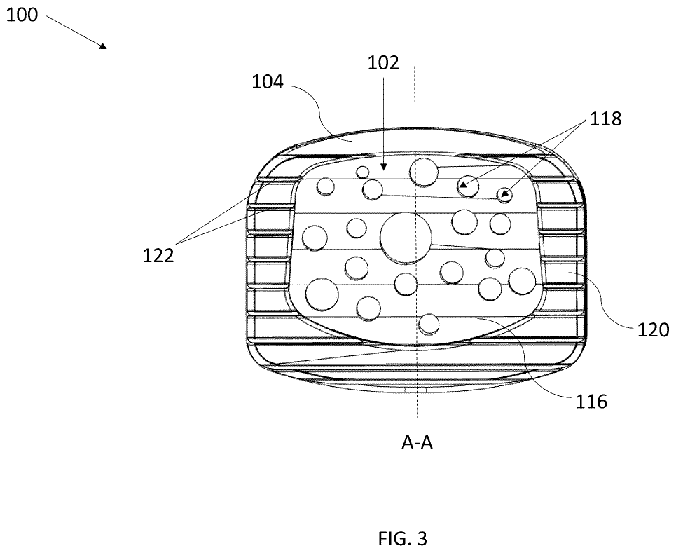

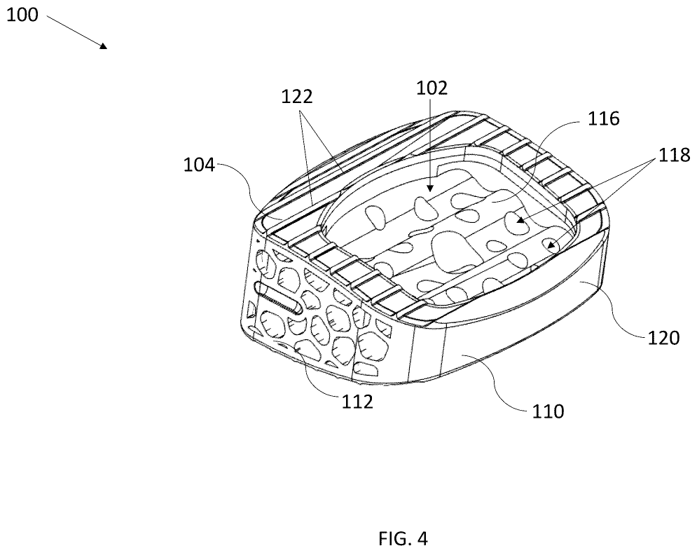

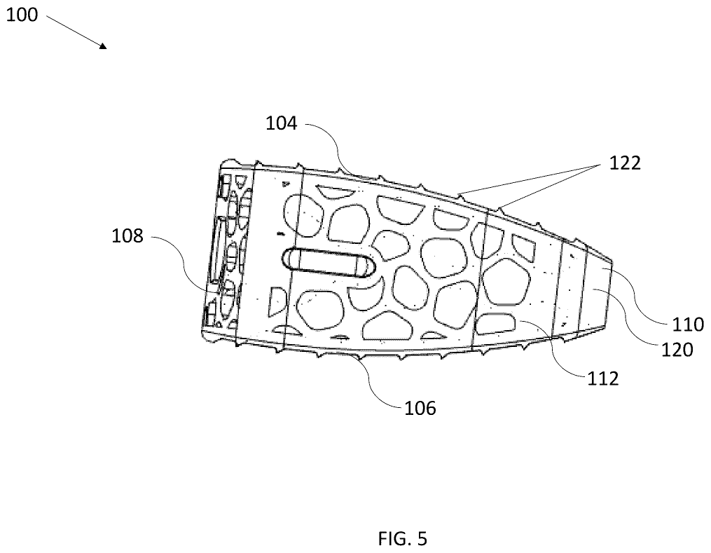

FIGS. 1-7 depict an IBD 100 in accordance with an embodiment of the present disclosure. IBD 100 is a spinal implant with an undulated crossbeam 116 spanning graft chamber 102 to secure biological graft material above and/or below crossbeam 116, as discussed in detail below. As shown, in addition to crossbeam 116 and graft chamber 102, IBD 100 includes an outer wall 120 defining the perimeter of the implant, superior and inferior sides 104 and 106 with ridges 122, and two engagement holes 124a and 124b formed in a front surface of IBD 100. Although each defined by outer wall 120, for case of reference, an anterior side 108, a posterior side 110, a first lateral side 112, and a second lateral side of IBD 100 are also labeled.

Graft chamber 102 extends from superior side 104 to inferior side 106.

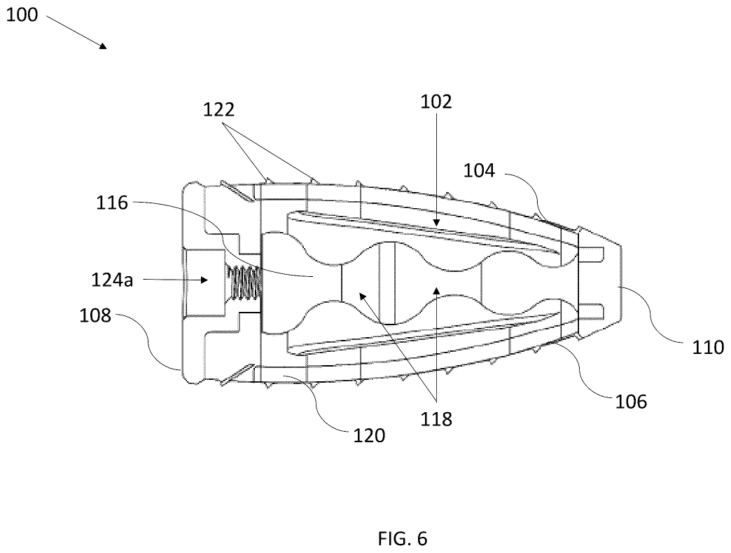

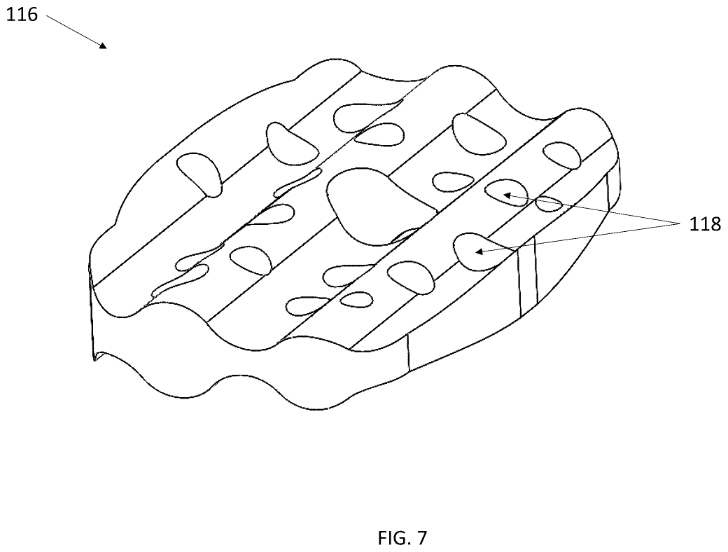

Crossbeam 116 is located within graft chamber 102 and is connected to anterior side 108, posterior side 110, first lateral side 112, and second lateral side (not shown). As best shown in FIGS. 6-7, crossbeam 116 has smooth, curved sinusoidal peaks that define a plurality of undulating surfaces. The peaks extend towards superior and inferior sides 104 and 106 and along a direction substantially parallel to anterior and posterior sides 108 and 110. Of course, in other embodiments (some of which are discussed more fully below), the crossbeam may be differently configured. For instance, the peaks could extend along a direction substantially parallel to lateral sides 112.

Biological graft material may be manually compressed into IBD 100 on both sides of crossbeam 116 facing superior and inferior sides 104 and 106 prior to inserting IBD 100 into a patient. As a result, biological graft material may also be at least partially compressed into channels 118. Since channels throughout an IBD in general typically only provide minimal frictional support for biological graft material, the undulations on crossbeam 116 prevent the material from dislodging. Specifically, the undulations on crossbeam 116 increase surface area and friction between the biological graft material and crossbeam 116, providing translational support to the biological graft material and therefore preventing the biological graft material from migrating to one side due to impaction upon insertion of IBD 100.

Biological graft material may be autologous and/or allogeneic bone graft, a bone growth enabling matrix, and/or bone growth stimulating substances. The bone growth enabling matrix may include endogenous bone forming cells (e.g., mesenchymal stem cells, osteoprogenitor cells, and osteoblasts) and osteoinductive and angiogenic growth factors. Additionally, the bone graft material may be a solid substance such as a sol-gel bioactive glass (e.g., silicate, borate, and borosilicate bioglasses) or sol-gel derived bone graft.

Though the embodiment of an IBD shown in the figures includes undulated surfaces on either side of the crossbeam, other embodiments may include an undulated surface on only one side of the crossbeam. Likewise, although the undulations are shown as smooth and curved sinusoidal peaks in this embodiment, the undulations can exhibit any shape necessary to cooperate with biological graft material. The surfaces of the crossbeam could in fact be designed differently depending on the specific type of bone growth material being utilized. Specifically, the crossbeam may exhibit irregular undulations, such as, but not limited to, different heights of peaks.

Channels 118 extend through crossbeam 116 in a substantially vertical direction between superior and inferior sides 104 and 106. Upon insertion of IBD 100, any biological graft material included in graft chamber 102 will permit osteogenic growth through the implant. Channels 118 are designed to not only receive some of such graft material, but also permit bone to grow through crossbeam 116. It is contemplated to also permit channels 118 to remain empty to facilitate cell transfer. In the embodiment shown, there are 20 channels 118 of differing diameters.

However, in other embodiments, there may be any number of channels.

Additionally, the channels may be any diameter. In some embodiments, the diameters of the channels may range from 1 to 100 mm and may preferably range from 30 to 50 mm. It is to be understood that the channels can be any geometry, shape, or dimension.

Outer wall 120 extends along a perimeter of anterior side 108, posterior side 110, first lateral side 112, second lateral side, and portions of superior and inferior sides 104 and 106. Outer wall 120 is porous on first lateral side 112, second lateral side, and posterior side 110 to permit osteogenic growth through the pores. The pores are of differing diameters. The pores may range from 100 to 1,000 μm and may preferably range from 100 to 600 μm. Additionally, the volume percent voids may have an average value of 55-65%. However, in other embodiments, the outer wall 120 may be solid with no porosity or, especially in the case of additive manufacturing of a solid wall, may have a porosity of substantially zero. Additionally, the outer wall 120 may be partially porous and may have any pore size.

Ridges 122 extend above superior side 104 and below inferior side 106. Ridges 122 are configured to engage vertebral bodies above and below IBD 100 and to prevent movement of IBD 100. In this embodiment, there are nine rows of ridges 122 extending between each lateral side 112. However, in other embodiments, there may be any number of ridges 122. Likewise, although the protrusions are shown as ridges 122 in this embodiment, the protrusions can exhibit any shape necessary to cooperate with vertebral bodies.

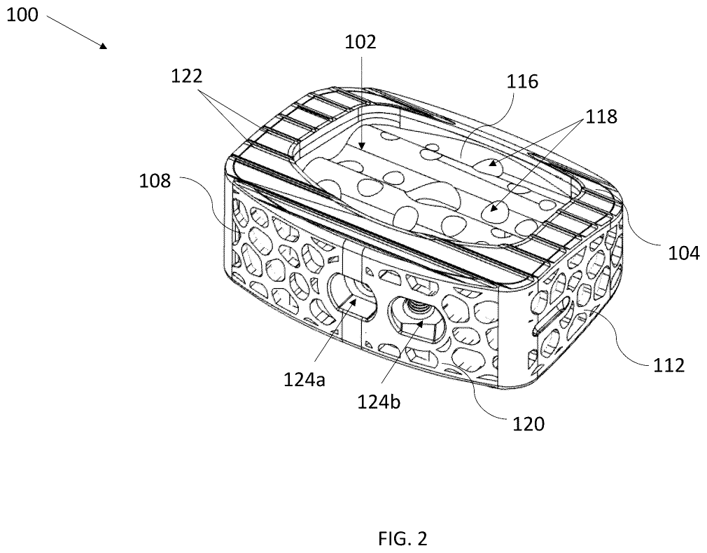

As best shown in FIG. 2, engagement holes 124a and 124b are located horizontally adjacent to each other in outer wall 120 on anterior side 108. Engagement holes 124a and 124b are configured to connect to insertion instruments (not shown) to insert IBD 100 into a patient. Specifically, engagement holes 124a and 124b may be connected to insertion instruments by a threaded connection, collet connection, or another connection known in the art.

As alluded to above, IBD 100 can be manufactured via a 3D printing or additive manufacturing process to include both solid and porous structures. For instance, the implant can be manufactured according to any of the methods disclosed in U.S. Pat. Nos. 7,357,664; 8,268,099; 8,268,100; 8,992,703; 9,456,901; 10,182,923; 10,398,559; 10,525,688; 10,716,673; 10,835,388; 11,000,386; 11,155,073; and 11,622,867, the disclosures of which are hereby incorporated by reference herein. Likewise, although specific embodiment implant structures are shown herein, IBDs according to the present invention may take on the form of any known spinal implant with the crossbeam being located in a graft chamber portion thereof. The designs could, for instance, be of any those disclosed in U.S. Pat. Nos. 8,382,767; 8,696,681; 8,801,721; 8,801,791; 9,095,385; 9,358,122; 9,392,673; 9,393,130; D824,518; 9,468,535; 9,480,577; 9,445,914; 9,867,713; 10,182,919; 8,425,529; 8,858,637; 11,173,047; 9,707,096; 10,292,832; 10,299,877; 10,327,908, 10,835,340; 11,173,041; 11,344,426; 9,987,051; 10,660,763; D824,518; 11,638,651; 10,271,958; 11,382,763; 8,801,721; 11,298,244; 9,585,762; 9,808,352; 10,004,608; 11,013,616; 11,491,028; 11,679,004; 10,299,877; 10,363,142; 10,441,430; 10,835,340; 10,610,374; 11,291,552; 11,331,200; 11,337,829; 11,612,496; 8,998,924; 9,987,149; 8,696,751; 8,454,695; 10,182,923; 10,548,738; 10,285,825; 10,835,388; 11,000,386; 10,398,559; 10,716,673; 11,622,867; and 11,173,047, the disclosures of which are hereby incorporated by reference herein.

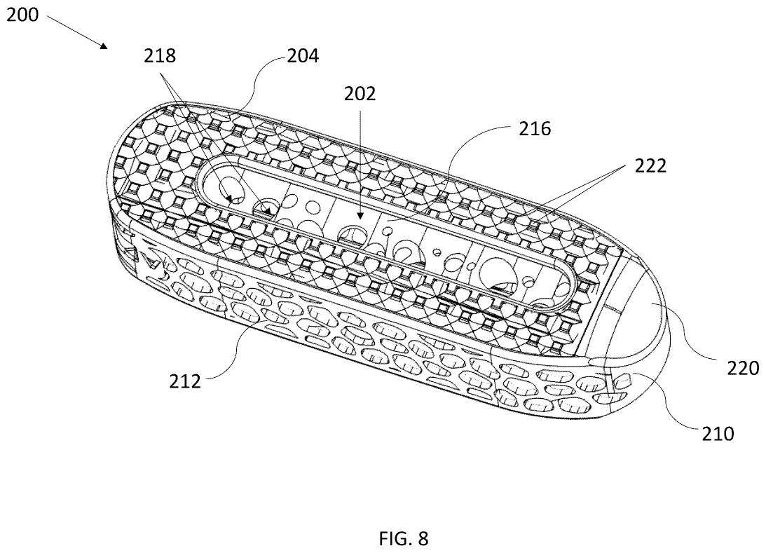

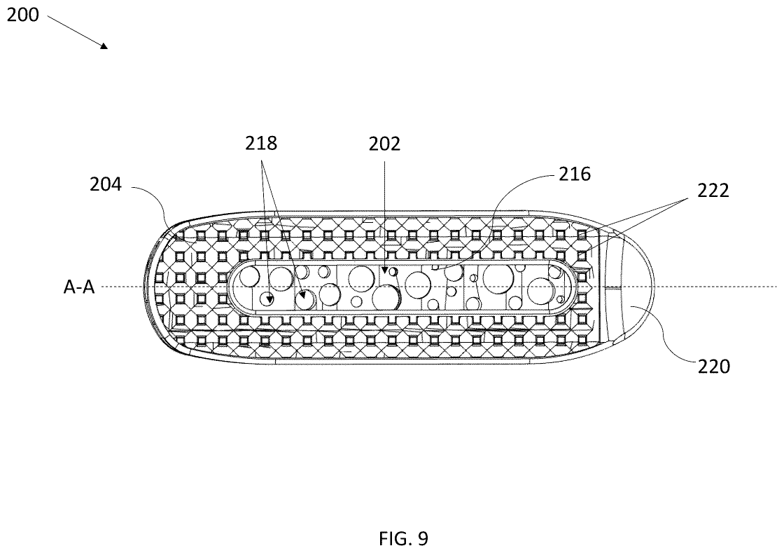

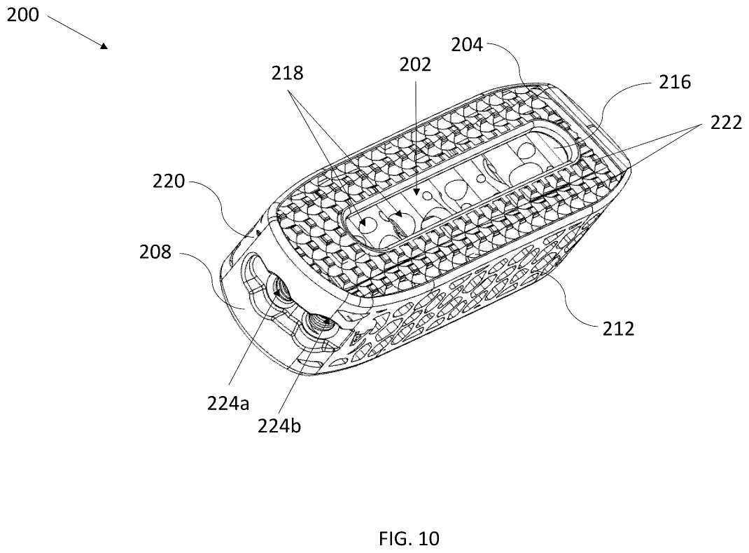

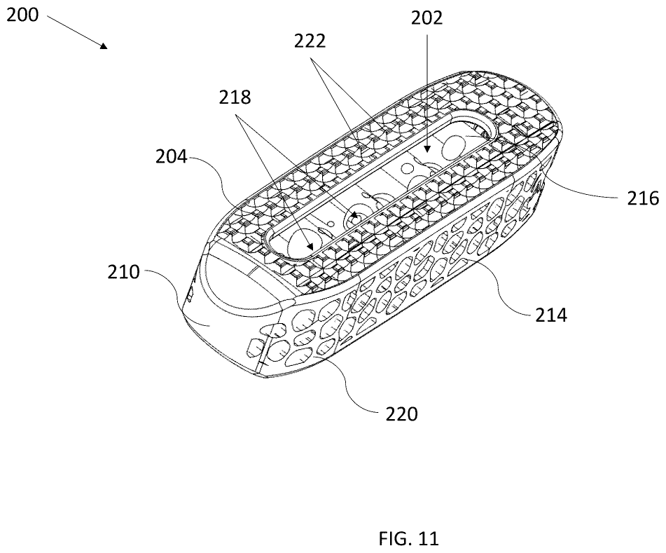

FIGS. 8-13 show an IBD 200 according to another embodiment of the present disclosure. In this embodiment, IBD 200 is similar to IBD 100 of FIGS. 1-7, and therefore like elements are referred to with similar numerals within the 200-series of numbers. The description of certain similar features between IBD 200 and IBD 100 is omitted for sake of brevity, and the following discussing focuses on the differences between the two implants.

IBD 200 differs from IBD 100 in that it is a lateral spinal implant with a greater length for insertion with a direct lateral transpsoas approach. Additionally, instead of ridges 122 on IBD 100, IBD 200 has a plurality of protrusions 222 on superior and inferior sides 104 and 106. Plurality of protrusions 222 are a frustopyrimidal shape. However, as stated above, the protrusions may exhibit any shape necessary to cooperate with vertebral bodies. Further, IBD 200 differs in that it has 24 channels 218 formed through crossbeam 216.

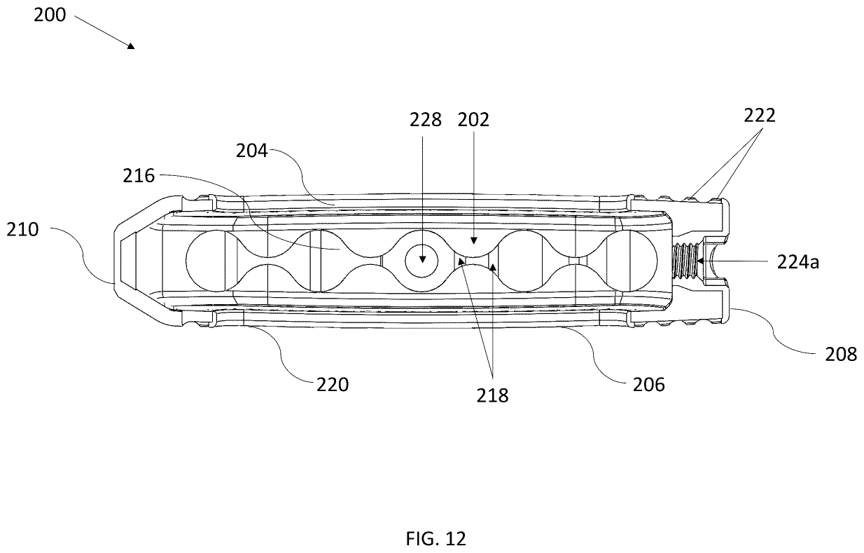

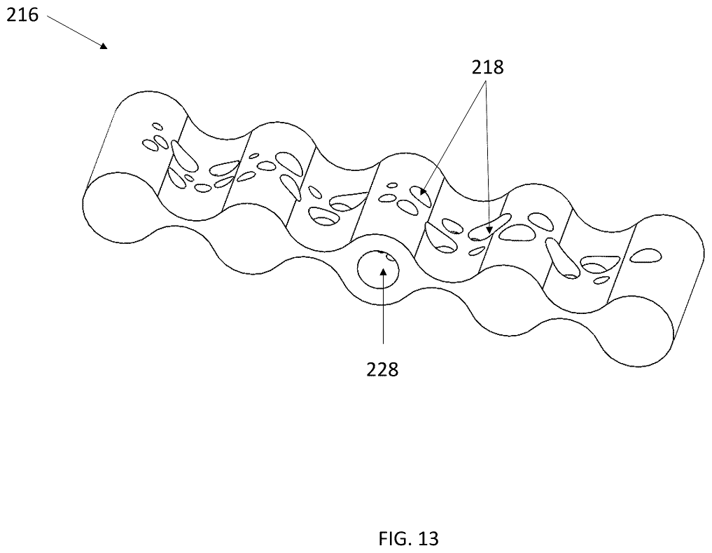

As best shown in FIGS. 12-13, IBD 200 also includes through hole 228 extending through lateral sides 212 and 214. Through hole 228 may be used for visual alignment under fluoroscopy to assess implant rotation during insertion and to therefore ensure that IBD 200 is properly placed within a patient. Specifically, through hole 228 will appear less dense than the surrounding porous and solid structures of IBD 200 in an X-ray image. Through hole 228 may range from 1 mm to 4 mm in diameter. However, it is to be understood that the through hole may be any diameter. A larger diameter of a through hole is easier to see on an X-ray image compared to a smaller diameter of a through hole.

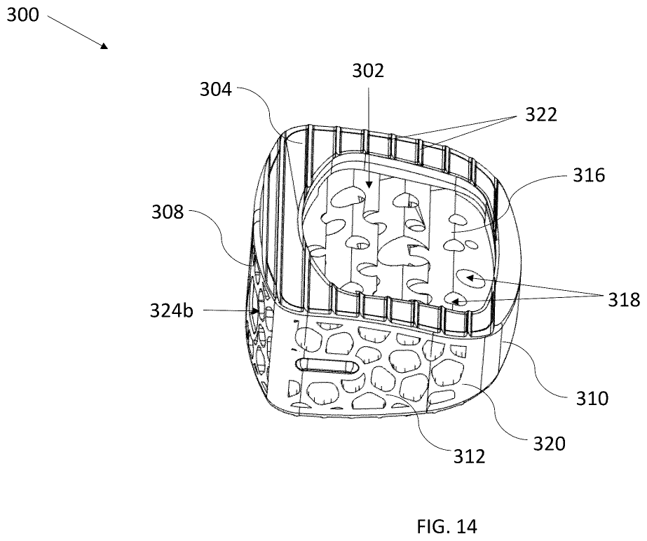

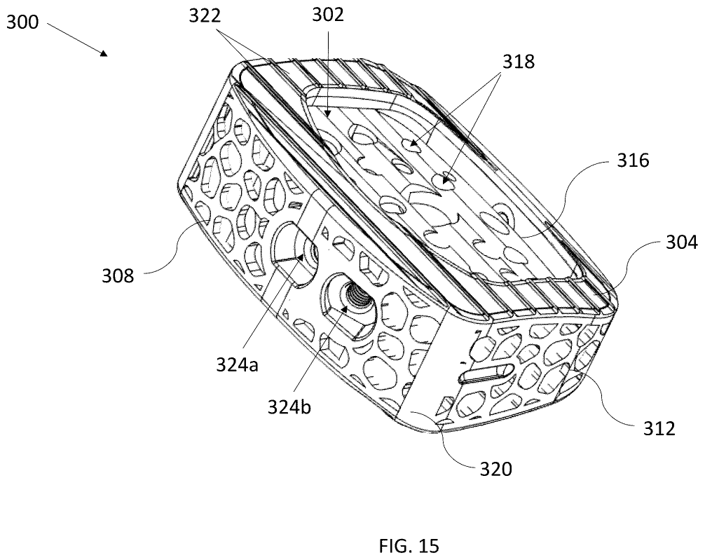

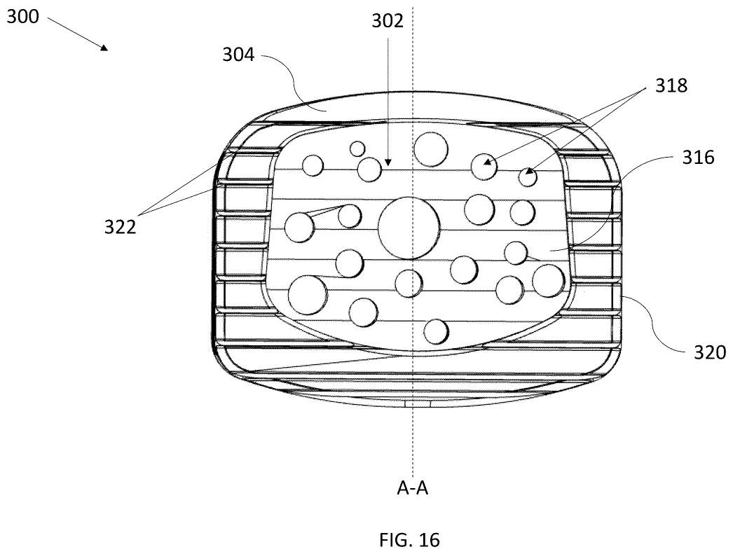

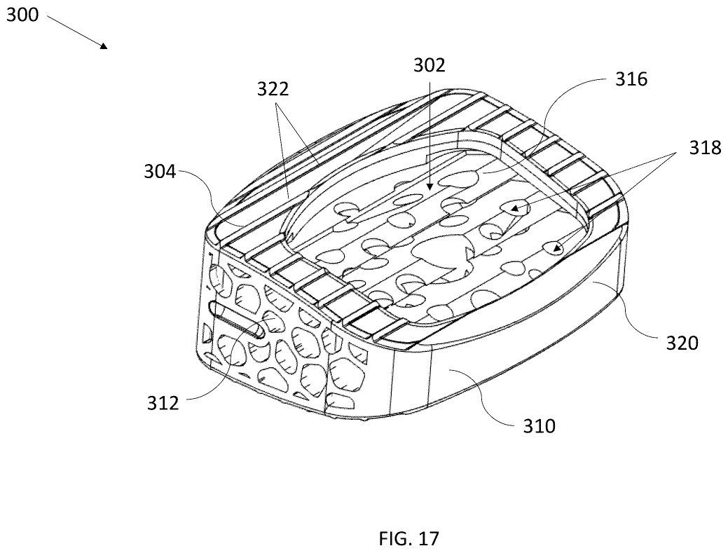

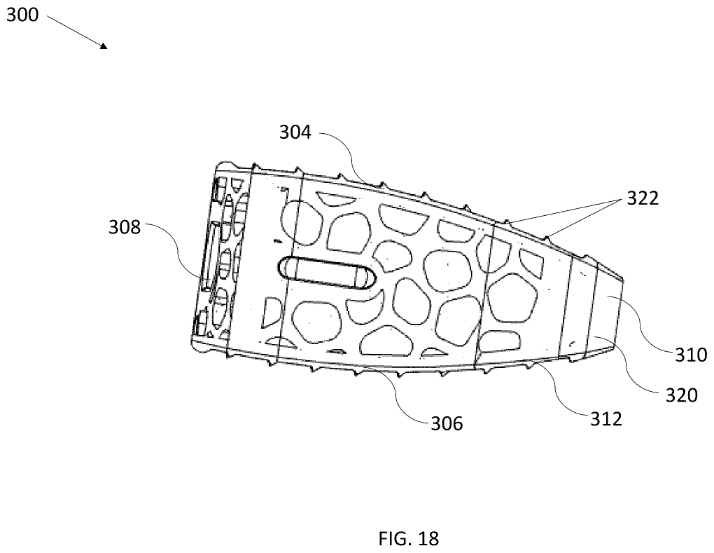

FIGS. 14-20 show an IBD 300 according to another embodiment of the present disclosure. In this embodiment, IBD 300 is similar to IBD 100 of FIGS. 1-7, and therefore like elements are referred to with similar numerals within the 300-series of numbers. Additionally, the description of certain similar features between IBD 300 and IBD 100 is omitted for sake of brevity, with the following again focusing on the differences between the two implants.

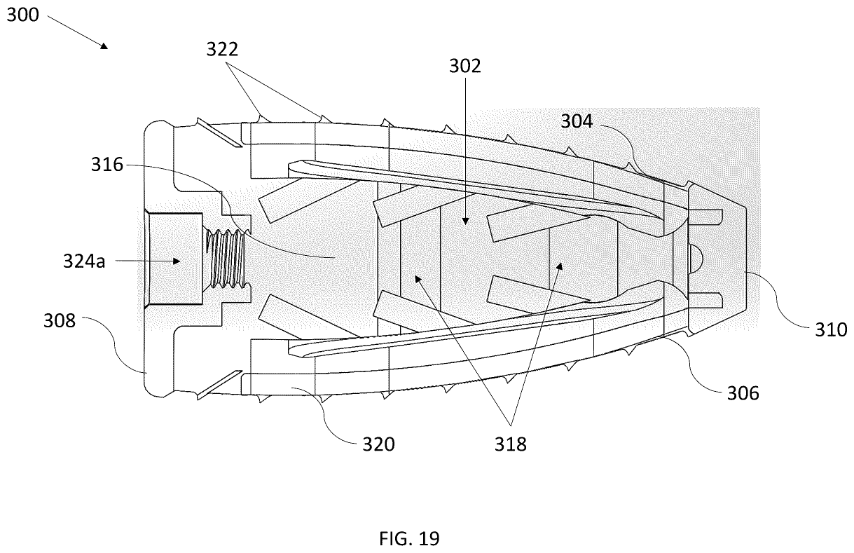

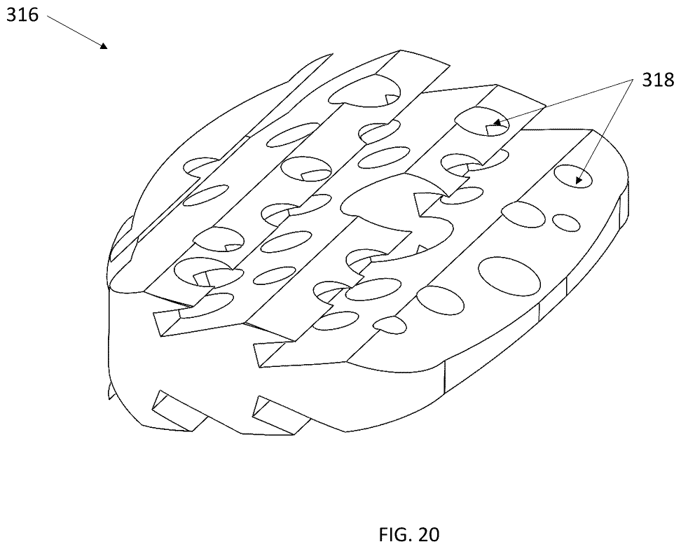

As best shown in FIGS. 19-20, IBD 300 differs from IBD 100 in that the undulations are a series of undercuts on a top and a bottom surface of crossbeam 116 that substantially face posterior side 310. The series of undercuts have sharp, planar sinusoidal peaks. Additionally, the series of undercuts face superior and inferior sides 104 and 106 so that impact to superior and inferior sides 104 and 106 upon insertion of IBD 300 into a disc space will push biological graft material horizontally and substantially parallel to superior and inferior sides 104 and 106 deeper into the series of undercuts. The movement of biological graft material into the series of undercuts is similar to movement of material through a net. Further, IBD 300 differs in that it has 20 channels 318 and eight rows of ridges 322 extending between each lateral side 312 and 314.

IBDs, and any other implant in accordance with the present invention, can be made of any material suitable for implanting into a human body, including, but not limited to, polymeric materials (e.g., PEEK) and metallic materials (e.g., stainless steel or titanium). IBDs according to the present invention can be manufactured utilizing any known process. While 3D printing and additive manufacturing are discussed above, it is contemplated to manufacture implants according to the present invention in any other known manner. For instance, it is contemplated to mold the implants, as well as form the implants bodies separately from the crossbeam. The crossbeam could be welded or otherwise fixedly attached to the implant body. Alternatively, it is contemplated to facilitate a removable connection between the crossbeam and implant body, thereby permitting the selective use of the crossbeam with a given implant.

Though the present disclosure is discussed in connection to spinal implants, the undulated crossbeam may be implemented in other types of medical implants. For example, this aspect can be implemented into implants like those utilized in tibial and other osteotomy procedures.

The disclosure set forth herein includes any possible combinations of the particular features set forth above, whether specifically disclosed herein or not. For example, where a particular feature is disclosed in the context of a particular aspect, arrangement, configuration, or arrangement, that feature can also be used, to the extent possible, in combination with and/or in the context of other particular aspects, arrangements, configurations, and arrangements of the technology, and in the technology generally.

Furthermore, although the technology herein has been described with reference to particular features, it is to be understood that these features are merely illustrative of the principles and applications of the present technology. It is therefore to be understood that numerous modifications, including changes in the sizes of the various features described herein, may be made to the illustrative arrangements and that other arrangements may be devised without departing from the spirit and scope of the present technology. In this regard, the present technology encompasses numerous additional features in addition to those specific features set forth in the claims below. Moreover, the foregoing disclosure should be taken by way of illustration rather than by way of limitation as the present technology is defined by the claims set forth below.

1. A spinal interbody device (IBD) comprising:
a graft chamber extending from a superior side of the IBD to an inferior side of the IBD; and
a crossbeam located within the graft chamber, the crossbeam having an undulated surface with a plurality of peaks facing at least one of the superior or inferior sides of the IBD.

2. The IBD of claim 1, wherein the plurality of peaks are configured to increase surface area and friction between the IBD and a biological graft material.

3. The IBD of claim 1, wherein the plurality of peaks are smooth and curved sinusoidal peaks.

4. The IBD of claim 1, wherein the plurality of peaks are sharp and planar sinusoidal peaks.

5. The IBD of claim 1, wherein the plurality of peaks are smooth and curved sinusoidal peaks and sharp and planar sinusoidal peaks.

6. The IBD of claim 1, further comprising a series of undercuts on a top and a bottom surface of the crossbeam, wherein the series of undercuts substantially face a posterior side of the IBD.

7. The IBD of claim 1, wherein the plurality of peaks are of differing heights.

8. The IBD of claim 1, wherein the plurality of peaks extend along a direction substantially parallel to an anterior side and a posterior side of the IBD.

9. The IBD of claim 1, wherein the plurality of peaks extend along a direction substantially parallel to a first and second lateral side of the IBD.

10. The IBD of claim 1, wherein the crossbeam has a plurality of channels extending from the undulated surface to a second surface of the crossbeam.

11. The IBD of claim 10, wherein the plurality of channels are of differing diameters.

12. A spinal interbody device (IBD) comprising:
a graft chamber; and
a crossbeam having two undulating surfaces facing opposing directions, the crossbeam extending from an anterior side of the graft chamber to a posterior side of the graft chamber.

13. The IBD of claim 12, wherein the opposing directions are towards a superior side of the IBD and an inferior side of the IBD.

14. The IBD of claim 12, wherein the crossbeam has a plurality of channels extending between the two undulated surfaces.

15. A spinal interbody device (IBD) comprising:
a graft chamber; and
a crossbeam having a first undulated surface facing a superior side of the IBD and a second undulated surface facing an inferior side of the IBD.

16. The IBD of claim 15, wherein the crossbeam has a plurality of channels extending through the first and second undulated surfaces.

17. The IBD of claim 15, further comprising biologic graft material in the graft chamber.

18. The IBD of claim 17, wherein the biologic graft material is between the first undulated surface and the superior side of the IBD.

19. The IBD of claim 17, wherein the biologic graft material is between the second undulated surface and the inferior side of the IBD.

20. The IBD of claim 15, further comprising an outer wall defining at least a portion of a perimeter of the IBD.
